(19) United States
(12) Patent Application Publication
(10) Pub. No.: US 2026/0054013 A1
(43) Pub. Date: Feb. 26, 2026

(54) SYRINGE SUPPORTING DEVICE

(71) Applicant: Alec WINTER, Austin, TX (US)

(72) Inventor: Alec WINTER, Austin, TX (US)

(21) Appl. No.: 19/308,849

(22) Filed: Aug. 25, 2025

Related U.S. Application Data
(60) Provisional application No. 63/686,199, filed on Aug. 23, 2024.

(51) Int. Cl. A61M 5/315; A61M 5/24

(52) U.S. Cl. CPC A61M 5/31505; A61M 5/2422

(57) ABSTRACT

A syringe supporting device includes a main body portion extending along an axis between a first end and a second end and defines a channel along the axis for receiving a barrel of the syringe. A vial housing is located at the first end of the main body portion and is configured to hold a vial. An adjustment member extends axially from the second end of the main body portion and is configured to receive a plunger of the syringe. The adjustment member defines a series of slots, each along a plane that is transverse to the axis. The slots are positioned in spaced relationship with one another and separated by tabs such that a handle of the plunger of the syringe is insertable into the slots into engagement with at least one of the tabs to inhibit movement of the plunger away from a retracted position.

### CROSS-REFERENCE TO RELATED APPLICATIONS

This utility patent application claims the benefit of and priority to U.S. provisional patent application Ser. No. 63/686,199, filed on Aug. 23, 2024, the entire disclosure of which is hereby incorporated by reference in its entirety.

### FIELD OF THE DISCLOSURE

The present disclosure relates to medical devices, and more specifically to syringes used for the administration of medications, such as testosterone replacement therapy.

### BACKGROUND

This section provides background information related to the present disclosure which is not necessarily prior art.

Syringes are known for administering liquid medications beneath the skin of recipients. Syringes typically include a channel barrel which terminates at a needle tip at a first end, and at an opening at a second end. A plunger is received in the opening and moveable within the barrel. During use, the needle tip is initially inserted into a rubber stopper at a top of a vial of the medication such that the needle tip is positioned in the liquid medication. The plunger is then pulled back to draw the medication into the barrel of the syringe.

A known issue with syringes, particularly when used to administer high viscosity liquid medications, like testosterone replacement therapy (TRT), is that the plunger can be biased toward returning to its initial, non-retracted position, especially when the plunger is quickly pulled back. This is primarily due to a resulting pressure difference between the chamber of the barrel relative to the chamber of the syringe. This can require a user to hold the plunger in its retracted position for a duration of time until the plunger settles into its retracted position and the barrel is satisfactorily filled with the medication. There remains a need for improvements to syringes.

### SUMMARY OF THE DISCLOSURE

According to an aspect of the disclosure, a syringe supporting device includes a main body portion that extends along an axis between a first end and a second end and defines a channel along the axis for receiving a barrel of the syringe. A vial housing is located at the first end of the main body portion and is configured to hold a vial in an inverted position. An adjustment member extends axially from the second end of the main body portion and is configured to receive a plunger of the syringe. The adjustment member defines a series of slots, each along a plane that is transverse to the axis. The slots are positioned in spaced relationship with one another and separated by tabs such that a handle of the plunger of the syringe is insertable into the slots into engagement with at least one of the tabs to inhibit movement of the plunger away from a retracted position.

According to another aspect of the disclosure, a method for securing a plunger of a syringe in an extended position includes providing a syringe supporting device that has a main body portion that extends along an axis between a first end and a second end. A vial housing is located at the first end of the main body portion. An adjustment member extends axially from the second end of the main body portion. The adjustment member defines a series of slots, each along a plane that is transverse to the axis. The slots are positioned in spaced relationship with one another by a plurality of tabs. The syringe supporting device further defines a channel that extends along the axis. The method also includes positioning a barrel of the syringe in the main body portion along the channel. The method also includes positioning a vial in the vial housing, with a needle of the syringe extending into the vial. The method also includes axially retracting a plunger of the syringe in the channel in the adjustment member such that a fluid in the vial is drawn into the barrel of the syringe. The method also includes inserting a handle of the plunger into one of the slots to inhibit axial movement of the plunger toward the tip of the syringe.

The plunger is secured in place by the slots, preventing it from being pulled back due to pressure differences between the vial and the barrel. This allows the barrel to fill with liquid medication while resting on a surface, freeing the user's hands and time. The supporting device is simply assembled and can be inexpensively mass-produced using methods like injection molding and 3D printing.

### BRIEF DESCRIPTION OF THE DRAWINGS

Other advantages of the present disclosure will be readily appreciated, as the same becomes better understood by reference to the following detailed description when considered in connection with the accompanying drawings wherein:

FIG. 1 is a front, perspective view of a first embodiment of a syringe supporting device, illustrating a syringe held by the syringe supporting device, and a plunger of the syringe in a retracted positions;

FIG. 2 is a front view of the first embodiment of the syringe supporting device and syringe;

FIG. 3 is another front, perspective view of the first embodiment of the syringe supporting device and syringe;

FIG. 4 is a front, perspective view of a second embodiment of the syringe supporting device;

FIG. 5 is a side view of the second embodiment of the syringe supporting device;

FIG. 6 is front view of the second embodiment of the syringe supporting device;

FIG. 7 is a rear view of the second embodiment of the syringe supporting device; and

FIG. 8 is a flow diagram of a method of using the syringe supporting device.

### DESCRIPTION OF THE ENABLING EMBODIMENT

Example embodiments will now be described more fully with reference to the accompanying drawings. In general, the subject embodiments are directed to a device for supporting a syringe and holding a plunger of the syringe in a retracted position to inhibit movement of the syringe back toward its initial position. However, the example embodiments are only provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. It should be appreciated that the teachings of the present disclosure could be applied to various types of vehicles such as recreation vehicles.

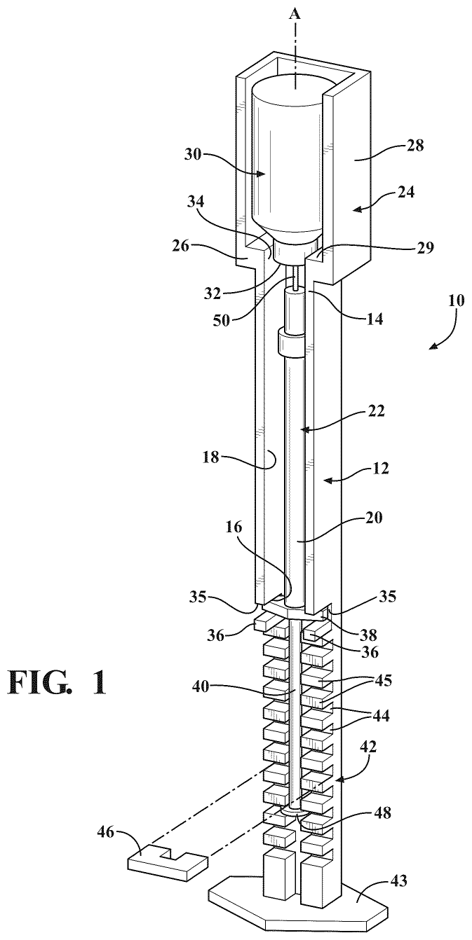

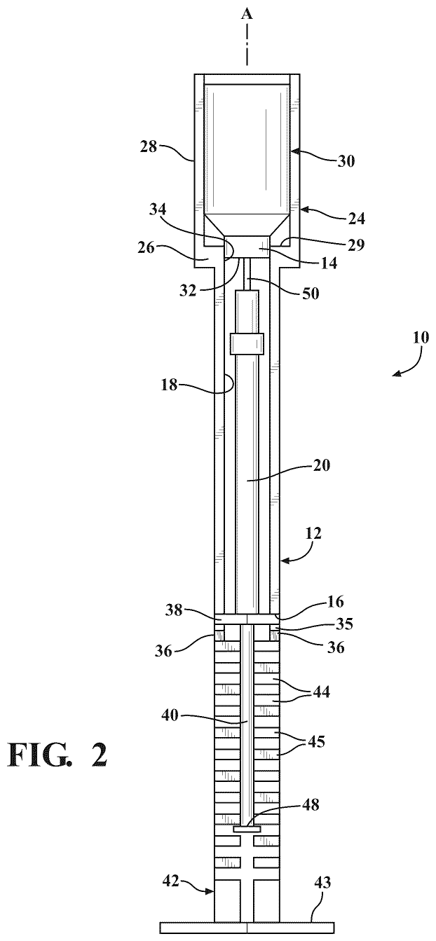

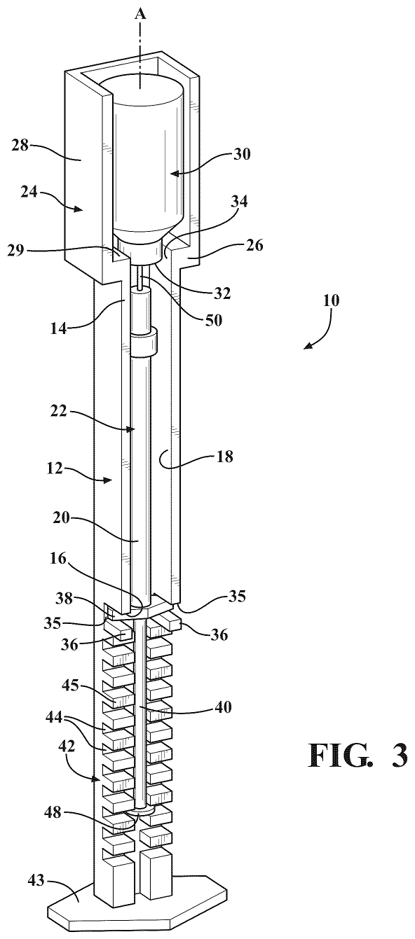

More particularly, referring to the figures, wherein like numerals indicate corresponding parts throughout the several views, embodiments of a supporting device 10, 210 for a syringe 22 are provided. With reference to FIG. 1, according to a first embodiment of the supporting device 10, it has an elongated main body portion 12 that extends along an axis A between a first (top) end 14 and a second (bottom) end 16. The main body portion 12 defines a portion of a channel 18 which is configured to receive a barrel 20 of a syringe 22. The main body portion 12 and associated channel 18 may have various sizes in order to accommodate differently sized syringes 22.

A vial housing 24 is located at the first end 14 of the main body portion 12. The vial housing 24 is generally cup-shaped with a bottom 26 and one or more sides 28 such that it is configured to support a vial 30 in an inverted position. As shown, while the vial 30 is in this position, a rubber stopper 32 of the vial 30 aligned with the channel 18 of the main body portion 12. The vial housing 24 may have any number of sides 28, and they may have various shapes. For example, a single tube-shaped side 28 may be used. The bottom 26 of the vial housing 24 defines an opening 34 into the channel 18 of the main body portion 12. According to other embodiments, the sides 28 of the vial housing 24 may taper inwardly for accommodating an associated taper on the vial 30. The vial housing 24 may have various sizes and shapes to accommodate different types of vials 30.

A pair of flanges 36 are located adjacent to the second end 16 of the main body portion 12. A gap 35 is defined axially between the second end 16 of the main body portion 12 and the pair of flanges 36. As shown in FIG. 1, the bottom end 16 of the main body portion 12 and the pair of flanges 36 are configured to support a rim 38 of the barrel 20 of the syringe 22, with the rim 38 positioned in the gap 35 to inhibit axial movement of the barrel 20 of the syringe 22 during retraction of a plunger 40 of the syringe 22 out of the barrel 20. As will be discussed in further detail below, according to embodiments, more pairs of flanges 36 may be present to define additional gaps 35 for receipt of the rim 38 of the barrel 20 of the syringe 22.

An adjustment member 42 extends axially downwardly beyond the flanges 36. The adjustment member 42 terminates at a stand 43 which extends radially outwardly for supporting the supporting device 10 and syringe 22 on a flat surface, like a table or countertop, with the vial 30 in the inverted position. The adjustment member 42 defines a series of slots 44, each along a plane that is transverse to the to the axis A. The slots 44 are separated from one another in spaced relationship with one another along an axial length of the adjustment member 42 via an arrangement of tabs 45.

The channel 18 is collectively defined along front surfaces of the vial housing 24, main body portion 12 and adjustment member 42. Specifically, a front portion of all of these features defines a gap along the channel 18. The channel 18 permits the vial 30 to be inserted into the vial housing 24, the barrel 20 of the syringe 22 to be inserted into the main body portion 12, and the plunger 40 to be inserted into the adjustment member 42.

As will be discussed in further detail below, during use, the barrel 20 of the syringe 22 is positioned in the channel 18 of the main body portion 12 of the supporting device 10, and the plunger 40 is axially moved into a retracted position to draw the contents of the vial 30 into the barrel 20 of the syringe 22 via a needle 50 that has punctured through the rubber stopper 32 of the vial 30. A handle 48 of the plunger 40 is then inserted into one of the slots 44 such that it engages one of the tabs 45 to inhibit movement of the plunger 40 back to its initial position. This prevents the plunger 40 from being unintentionally drawn back into the barrel 22.

According to embodiments, as illustrated in FIG. 1, a clip 46 may also be insertable into the slots 44 for further inhibiting movement of the handle 48 of the plunger 40 of the syringe 22 away from an extended position. The clip 46 is positioned in one of the slots 44 above the handle 48 of the plunger 40.

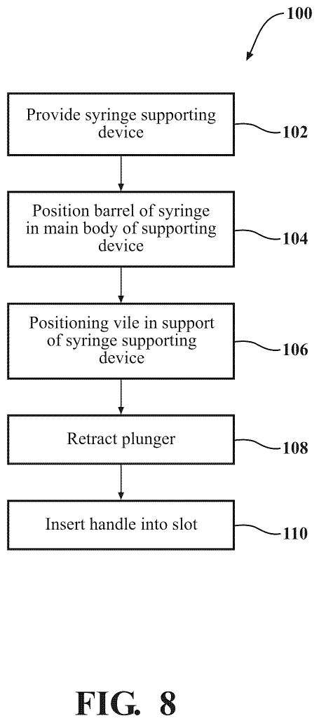

FIG. 8 presents a method 100 for securing a plunger 40 of a syringe 22 in an extended position. The method first includes 102 providing a supporting device 10 in accordance with the above. The method continues with 104 positioning a barrel 20 of the syringe 22 in the main body portion 12 of the supporting device 10. The method also includes 106 positioning a vial 30 in the vial housing 24 of the supporting device 10, with the needle 50 of the syringe 22 extending into the vial 30. More particularly, the needle 50 punctures through the rubber stopper 32 of the vial 30 to access the liquid medication in the vial 30.

The method continues with 108 axially retracting the plunger 40 of the syringe 22 such that the liquid medication in the vial 30 is drawn into the barrel 20 of the syringe 22. The method then includes 110 positioning the handle 48 of the plunger 40 into one of the slots 44 of the main body portion 12 to axially secure the plunger 40 in place. The handle 48 may be inserted in any of the slots 44 depending on how far the plunger 40 is retracted. In embodiments which include a clip 46, the method may include 112 inserting the clip 46 into one of the slots 44 above the handle 48 to further secure the handle 48 and plunger 40 in place.

Because the plunger 40 is held in place via the slots 44, the plunger 40 is prevented from being drawn back into its original position due to a pressure difference between the inside of the vial 30 and the inside of the barrel 20, and the barrel 20 is able to fill with the liquid medication while sitting on a supporting surface via the stand 43, without the user holding the plunger 40 in place in its drawn position. This frees the user's time and hands.

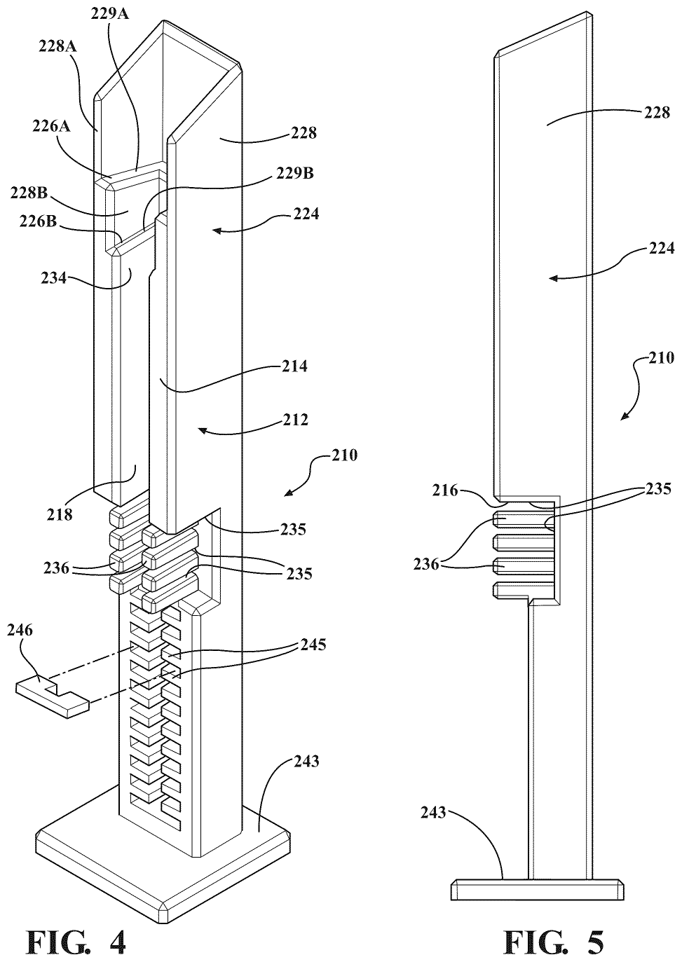

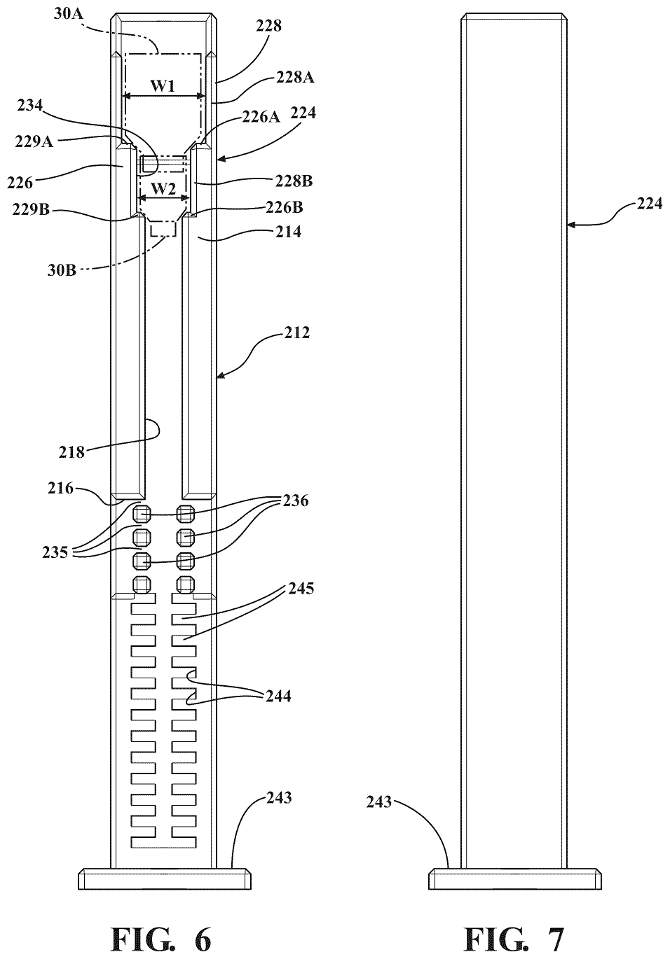

FIGS. 4-7 present a second embodiment of the supporting device 210. According to this embodiment, the vial housing 224 has both an upper bottom surface 226A and a lower bottom surface 226B in axially spaced relationship with one another. The vial housing 224 also has an upper side 228A which extends upwardly from the upper bottom surface 226A, and a lower side 228B which extends upwardly from the lower bottom surface 226B to the upper bottom surface 226A. As shown in FIG. 6, a width W1 (perpendicular to the axis A) of the compartment of the vial housing 224 along the upper side 228A is larger than a width W2 of the compartment along the lower side 228B to define upper and lower shelves 229A, 229B along the upper and lower bottoms 226A, 226B such that the vial housing 224 is configured to hold vials 30A, 30B of multiple sizes. This permits the supporting device 210 to be used for a variety of different medications with vials 30A, 30B of different widths, thus providing flexibility in use.

Also according to this embodiment, a plurality of pairs of flanges 236 are provided, with the flanges 326 spaced axially from one another by a plurality of gaps 235, which are each configured to receive the rim 38 of the plunger 40. This permits the supporting device 210 to receive and accommodate syringes 22 of various lengths, thus providing flexibility in use.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in that particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or later, or intervening element or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to described various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in any embodiment, even if not specifically shown or described. The same may also be varied in many ways. Such variations are not to be regarded as a departure from the disclosure, and all such modifications are intended to be included within the scope of the disclosure.

What is claimed is:

1. A syringe supporting device, comprising:
a main body portion extending along an axis between a first end and a second end and defining a channel along the axis for receiving a barrel of the syringe;
a vial housing at the first end of the main body portion configured to hold a vial in an inverted position;
an adjustment member extending axially from the second end of the main body portion and configured to receive a plunger of the syringe; and
the adjustment member defining a series of slots, each along a plane being transverse to the to the axis, the slots positioned in spaced relationship with one another and separated by tabs such that a handle of the plunger of the syringe is insertable into the slots into engagement with at least one of the tabs to inhibit movement of the plunger away from a retracted position.

2. The syringe supporting device as set forth in claim 1, wherein the tabs are comprised of a plurality of pairs of the tabs each aligned with one another along a plane, and wherein the tabs of each of the pairs of tabs are separated from one another by the channel.

3. The syringe supporting device as set forth in claim 1, wherein the pairs of tabs are separated from one another in the axial direction by the slots.

4. The syringe supporting device as set forth in claim 1, further including at least one flange located adjacent to the second end of the main body portion, wherein at least one gap is located between the at least one flange and the second end of the main body portion, the at least one gap for receiving a rim of a barrel of the syringe with the rim engaging the at least one flange to inhibit axial movement of the barrel.

5. The syringe supporting device as set forth in claim 1, wherein the at least one flange includes a plurality of pairs of flanges, wherein the flanges of each of the pairs of flanges are separated from one another by the channel, and wherein the pairs of flanges are separated from one another axially by a plurality of gaps such that the rim of the barrel of the syringe may be inserted into any of the plurality of gaps.

6. The syringe supporting device as set forth in claim 1, wherein the vial housing is generally cup-shaped with at least one bottom surface and at least one side extending upwardly from the sides for supporting the vial in an inverted position against the bottom and between the sides, and wherein the bottom partially defines the channel for receiving the syringe.

7. The syringe supporting device as set forth in claim 1, wherein the sides of the vial housing taper radially inwardly to the bottom for accommodating an associated taper of the vial.

8. The syringe supporting device as set forth in claim 6, wherein the vial housing defines at least one shelf along the bottom surface and the channel for supporting the vial.

9. The syringe supporting device as set forth in claim 8, wherein the at least one bottom of the vial housing includes an upper bottom surface and a lower bottom surface being axially spaced from one another, wherein the at least one side of the vial housing includes at least one upper side extending axially upwardly from upper bottom surface and a lower side extending upwardly from the lower bottom surface to the upper bottom surface, wherein the upper side extends outwardly to a greater extent that then lower side to define a lower shelf between the lower bottom surface and the channel, and an upper shelf between the upper bottom surface and the lower side such that the upper and lower shelves are configured to receive vials of different sizes.

10. The syringe supporting device as set forth in claim 2, further including a clip insertable into the slots axially between the tabs for further securing plunger in place at a desired axial location.

11. The syringe supporting device as set forth in claim 1, further including a stand connected to the adjustment member opposite the main body portion, the stand extending radially outwardly for supporting the supporting device and syringe on a supporting surface.

12. The syringe supporting device as set forth in claim 1, wherein the vial housing has a bottom segment extending perpendicularly to the axis and sides extending axially upwardly from the bottom segment, and wherein the vial housing defines at least one shelf between channel and the bottom segment.

13. A method for securing a plunger of a syringe in an extended position, comprising:
providing a syringe supporting device having a main body portion extending along an axis between a first end and a second end, a vial housing at the first end of the main body portion, an adjustment member extending axially from the second end of the main body portion, the adjustment member defining a series of slots, each along a plane being transverse to the axis, the slots positioned in spaced relationship with one another by a plurality of tabs, the syringe supporting device further defining a channel extending along the axis;
positioning a barrel of the syringe in the main body portion along the channel;
positioning a vial in the vial housing, with a needle of the syringe extending into the vial;
axially retracting a plunger of the syringe in the channel in the adjustment member such that a fluid in the vial is drawn into the barrel of the syringe; and
inserting a handle of the plunger into one of the slots to inhibit axial movement of the plunger toward the tip of the syringe.

14. The method as set forth in claim 13, wherein the tabs are comprised of a plurality of pairs of the tabs, wherein the tabs of each of the pairs are aligned with one another along a plane and separated from one another by the channel.

15. The method as set forth in claim 14, wherein the pairs of tabs are axially separated from one another by the slots.

16. The method as set forth in claim 13, further including at least one flange located adjacent to the second end of the main body portion, wherein at least one gap is located between the at least one flange and the second end of the main body portion, the at least one gap for receiving a rim of a barrel of the syringe with the rim engaging the at least one flange to inhibit axial movement of the barrel.

17. The method as set forth in claim 16, wherein the at least one flange includes a plurality of pairs of flanges, wherein the flanges of each pair of flanges are separated from one another by the channel, and wherein the pairs of flanges are separated from one another axially by a plurality of gaps such that the rim of the barrel of the syringe may be inserted into any of the plurality of gaps.

18. The method as set forth in claim 13, wherein the vial housing is generally cup-shaped with a bottom surface and sides extending upwardly from the sides for supporting the vial in an inverted position against the bottom surface and between the sides, and wherein the bottom surface partially defines the channel for receiving the syringe.

19. The method as set forth in claim 13, wherein the sides of the vial housing taper radially inwardly to the bottom surface for accommodating an associated taper of the vial.

20. The method as set forth in claim 13, further including a clip insertable into the slots between the tabs for further securing plunger in place at a desired axial location.
